(19) United States
(12) Plant Patent Application Publication
(10) Pub. No.: US 2025/0338783 P1
(43) Pub. Date: Oct. 30, 2025

(54) SEASHORE PASPALUM PLANT 'UGP 73'

(50) ;

(71) Applicant: University of Georgia Research Foundation, Inc., Athens, GA (US)

(72) Inventors: Paul Raymer, Milner, GA (US); Brian Schwartz, Tifton, GA (US)

(21) Appl. No.: 19/181,402

(22) Filed: Apr. 17, 2025

Related U.S. Application Data
(60) Provisional application No. 63/731,363, filed on Apr. 24, 2024.

(51) Int. Cl. A01H 6/46; A01H 5/00

(52) U.S. Cl. CPC A01H 6/46; A01H 5/00
USPC PLT/388

(57) ABSTRACT

A new and distinct cultivar of Seashore Paspalum plant, botanically known as Paspalum vaginatum Swartz, named ‘UGP 73’, characterized by its excellent turf quality, excellent turf color, superior drought tolerance, superior salt tolerance and better tolerance to shade than other Paspalum vaginatum cultivars.

Genus and species: Latin name: Paspalum vaginatum Swartz.

Variety denomination: Variety denomination: The new Paspalum vaginatum Swartz claimed is of the cultivar denominated ‘UGP 73’.

### BACKGROUND OF THE INVENTION

The present invention relates to a new and distinct cultivar of Seashore Paspalum plant, botanically known as Paspalum vaginatum Swartz, and hereinafter referred to by the cultivar name ‘UGP 73’.

UGP 73 is a vegetative clone developed from a single F1 seed arising from the hand-pollinated cross (Durban (PI 614678) X G05-1743). This cross was made in the greenhouse at Griffin, Georgia in 2007 by Dr. Paul Raymer. The female parent, Durban, was collected at the Durban Country Club in South Africa.

‘UGP 73’ is a vigorous vegetative seashore paspalum cultivar that produces excellent quality turf under a range of mowing heights, and is suitable for use on golf courses, athletic fields, and other recreational venues as fine turf. ‘UGP 73’ has been under development and evaluation for 16 years. ‘UGP 73’ has been evaluated extensively in both in the National Turfgrass Evaluation Program (NTEP) at eight southern locations from 2016-2020 and as part of a USDA Specialty Crops Research Grant (SCRI) at six southern locations from 2011-2013 and in SCRI advanced trials at six locations in 2016-2019. In the two-year SCRI trial, ‘UGP 73’ ranked 2^{nd} in overall turf quality out of 80 entries and ranked 1^{st} in turf quality under non-drought conditions.

### BRIEF SUMMARY OF THE INVENTION

The new Paspalum vaginatum Swartz cultivar ‘UGP 73’ has not been observed under all possible environmental conditions. The phenotype may vary somewhat with variations in environment and cultural practices such as temperature, water and fertility levels, soil types, and light intensity without, however, any variance in genotype.

The following traits have been repeatedly observed and are determined to be the unique and distinguishing characteristics of the new Paspalum cultivar named ‘UGP 73’. In combination, these traits set ‘UGP 73’ apart from all other existing varieties of Paspalum known to the inventors:
1. ‘UGP 73’ has excellent turf quality over a wide range of mowing heights and has often outperformed Salam™, ‘SeaIsle 1’, SeaStar®, Platinum TE®, and SeaDwarf™ (Tables 1, 7, 11, and 12).
2. ‘UGP 73’ has excellent turf color, often better than Salam™, ‘SeaIsle 1’, SeaStar®, Platinum TE®, and SeaDwarf™ (Tables 2, 5, 11, and 12).
3. ‘UGP 73’ has demonstrated superior drought tolerance to SeaStar® over a wide range of irrigation regimes (Table 7).
4. The salt tolerance of ‘UGP 73’ is similar to that of SeaStar® and better than that of ‘SeaIsle 1’ (Table 10).
5. ‘UGP 73’ has demonstrated better tolerance to 60% shade than has Platinum TE® and SeaStar® (Table 9).

Additional characteristics of ‘UGP 73’ known to the inventors also include:
1. Good turf density (Table 3).
2. Acceptable retention of color and quality during the fall and winter months following light frost or cool temperatures (Tables 4 and 5).
3. Medium to fine leaf texture.
4. Good resistance to dollar spot disease, Clarireedia sp. (Tables 6, 11, and 12)
5. Produces acceptable quality sod with good tensile strength (Table 8).

### BRIEF DESCRIPTION OF THE DRAWINGS

The accompanying colored photographic illustrations show the overall appearance and distinct characteristics of the new Paspalum vaginatum Swartz cultivar denominated ‘UGP 73’ showing the colors as true as possible. Colors in the photographs may differ slightly from the color values cited in the detailed botanical description, which accurately describes the colors of the new Genus species ‘cultivar name’.

FIG. 1 shows a potted greenhouse sample of ‘UGP 73’.

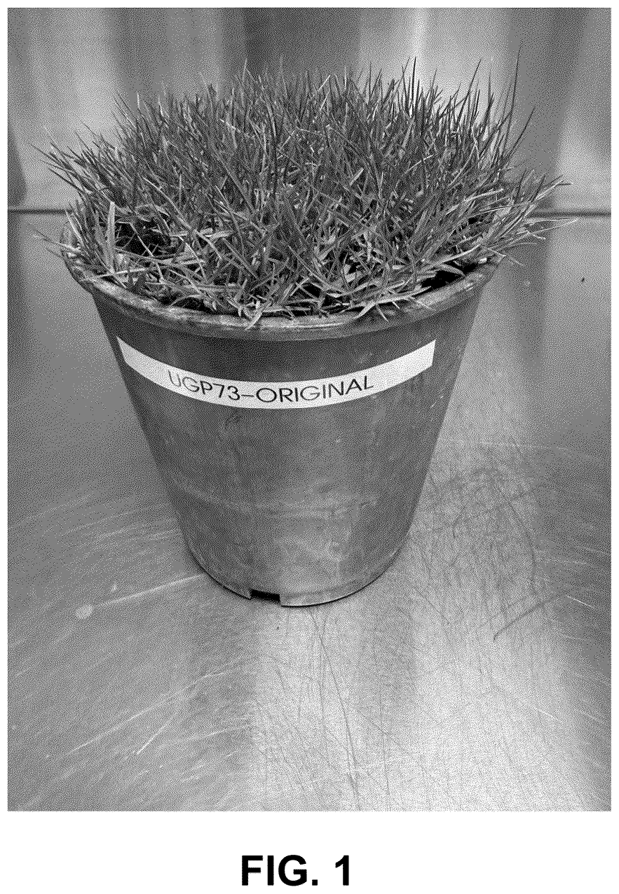

FIG. 2 shows abundance of stolons and rhizomes in a greenhouse flat of ‘UGP 73’.

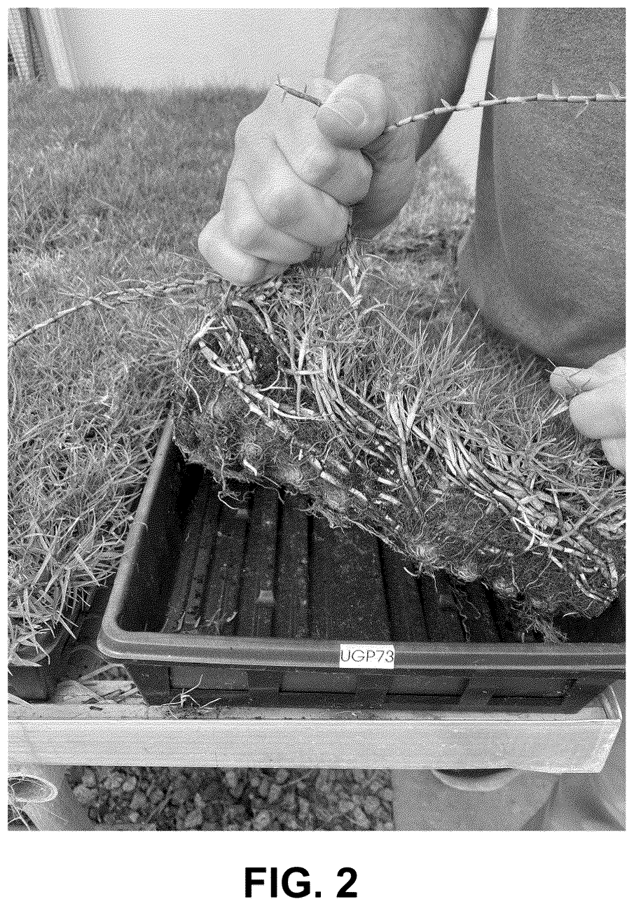

FIG. 3 shows a field of ‘UGP 73’ as managed turf.

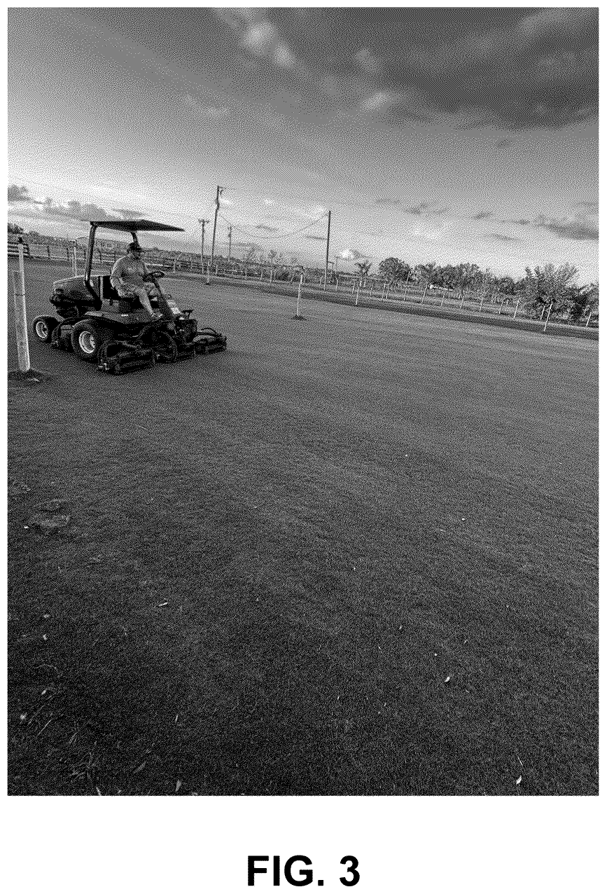

FIG. 4 shows ‘UGA 73’ in use on a coastal landscape.

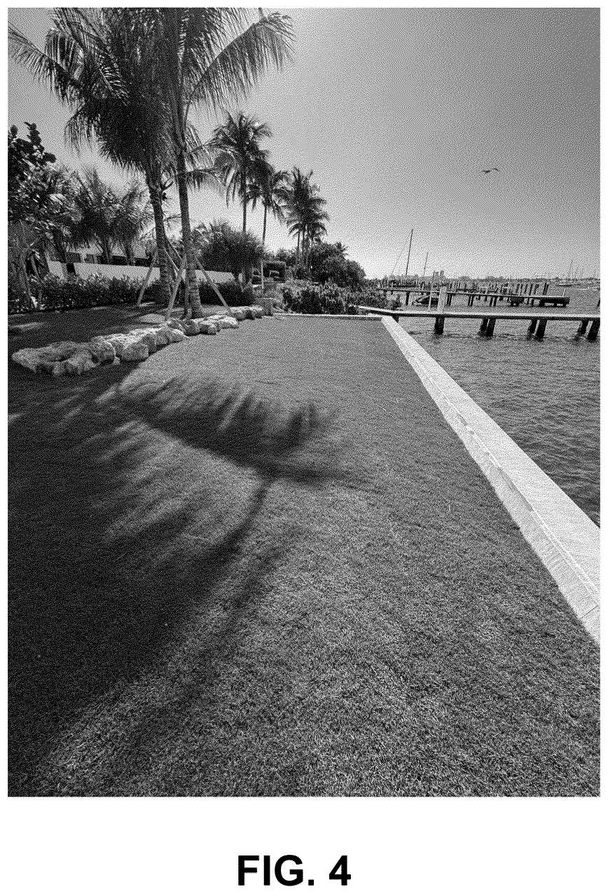

FIG. 5 compares the SSR Pv53 generated banding patterns obtained from DNA of ‘UGP 73’ to that from commercial cultivars.

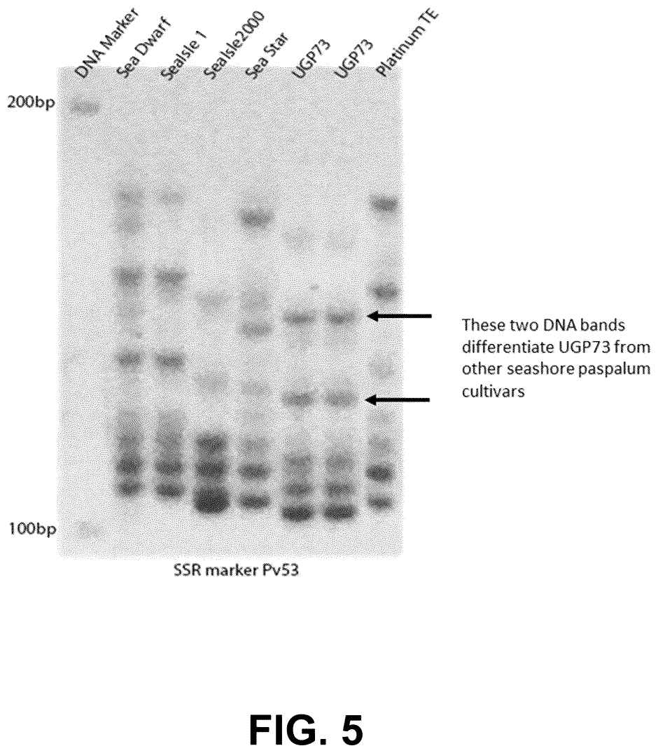

### DETAILED BOTANICAL DESCRIPTION

The following traits have been consistently observed in the original plant of this new variety grown in Griffin, Georgia, and, to the best knowledge of the inventors, their combination forms the unique characteristics of the new variety ‘UGP 73’.

Certain characteristics of this variety, such as growth and color, may change with changing environmental conditions (e.g., light, temperature, moisture, nutrient availability, or other factors). Color descriptions and other terminology are used in accordance with their ordinary dictionary descriptions, unless the context clearly indicates otherwise. Color designations are made with reference to The Royal Horticultural Society (R.H.S.) Colour Chart.

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society, London, England in 2007, except where general terms of ordinary dictionary significance are used.

The aforementioned photographs and following observations, measurements, and values describe plants of the Paspalum vaginatum Swartz cultivar ‘UGP 73’. Data were collected from plants that were approximately eight years old and were grown in the ground under greenhouse and outdoor conditions in Griffin, GA. The average outdoor low temperatures ranged from lows of about 33° F. to 42° F. in the winter, to average high temperatures of about 85° F. to 92° F. in summer.
1. The plant: ‘UGP 73’ is a vigorous variety with upright growth habit. It is dense and spreads rapidly by producing large numbers of rhizomes and stolons. The mean height of the unmown canopy (culm height) is 16.4 cm, with a seed head exsertion height above the canopy of 4.8 cm.
2. Flowering tillers: The mean length and diameter of the peduncle are respectively 9.6 mm and 0.6 mm. The mean length of the longest spike is 23.7 mm and there are two spikes per inflorescence. The mean length of the spike branch from the flag leaf is 8.9 mm and the length and width of the blade on the flag leaf are respectively 6.0 mm and 0.9 mm. The mean length of the sheath on the flag leaf is 33.3 mm. The mean length and width of the blade of the 4th leaf (the flag leaf is the 1^{st} leaf) are respectively 37.4 mm and 3.1 mm, and the mean length of the sheath on the 4^{th} leaf is 14.1 mm. The mean length of the 4^{th} internode is 11.3 mm. The color of the upper leaf surface is Green 137 B and the lower leaf surface color is Green 137 C. The anther color is Violet 83 A, and stigma color is Violet 86 A. Seeds are not produced in pure monostands due to self-incompatibility.
3. Stolons: The mean length and diameter of the 4^{th} internode from the tip are 13.8 mm and 1.6 mm respectively. The mean length and width of the 4^{th} leaf blade, at the 4^{th} node on stolon, are 6.7 mm and 2.6 mm respectively, with a leaf sheath that is 8.6 mm long. The color of the stolon is Yellow-Green 146 D.
4. Method of propagation: Vegetative (clonal)
5. Pedigree and history: UGP 73 is a vegetative clone developed from a single F1 seed arising from the hand-pollinated cross (Durban (PI 614678) X G05-1743). This cross was made in the greenhouse at Griffin, Georgia in 2007 by Dr. Paul Raymer. The female parent, Durban, was collected at the Durban Country Club in South Africa. Originally designated as G07-033.4, the single plant was germinated from seed in the laboratory and was established in the greenhouse. The seedling was visually screened for acceptable turf quality prior to being transplanted into the field for initial turf evaluation in the spring of 2010. G07-033.4 was evaluated in un-replicated field plots (2010 single plant nursery) mowed at 1.5 inches height of cut (h.o.c.) along with approximately 2000 other seedlings beginning in June of 2010. G07-033.4 was one of 80 lines selected for further evaluation in the fall of 2010 based on turf superior quality, density, color, and resistance to dollar spot disease. G07-033.4 was clonally propagated and increased during the spring of 2011 and included in a group of 80 seashore paspalum lines and re-designated as UGP73 in replicated trials at seven southern field locations (Griffin, GA; Tifton, GA; College Station, TX; Dallas, TX; Raleigh, NC; Gainesville, FL; and Stillwater, OK.) for turf performance and drought tolerance as part of a USDA Specialty Crops Research Grant (SCRI). In this two-year evaluation over seven locations, UGP73 ranked 2^{nd} in overall turf quality out of 80 entries and ranked 1^{st} in turf quality under non-drought conditions (data not presented). In 2015, UGP 73 was increased clonally in the greenhouse for inclusion in the National Turfgrass Evaluation Program (NTEP) seashore paspalum trials at eight southern locations from 2016-2020. NTEP trial locations were Auburn, AL; Fayetteville, AR; Riverside, CA; Jay, FL; Griffin, GA; Mississippi State, MS; Stillwater, OK; and College Station, TX and (h.o.c.) at these locations ranged from 0.5-2.5 inches. UGP 73 was also included in extensive trials associated with our second federal SCRI grant (2016-2019) where it was further evaluated for drought tolerance, salt tolerance, shade tolerance, and sod strength. In 2019, UGP 73 one of 23 advanced breeding lines and four standard cultivars included in a “greens” trial (0.110 to 0.175 inch h.o.c.). After the failure of an advanced line fairway trial (established in 2017) on the Griffin Campus, UGP 73 was included as one 28 entries in an advanced fairway trial established in 2020 on the UGA Griffin campus. Foundation and Registered fields of UGP 73 are established at Creekside Growers, Arcadia, Florida.
6. Description of plant material: UGP 73 is a vigorous clonal line that produces excellent quality turf under a range of mowing heights (Tables 1, 8, 12, and 13) and is suitable for use on golf courses, athletic fields, and other recreational venues as a fine turf. UGP 73 has excellent turf color (Tables 2, 5, 12, and 13), good turf density (Table 3) and acceptable retention of color and quality during the fall and winter months following light frost or cool temperatures (Tables 4 and 5). In limited evaluations, UGP 73 has shown less winter kill than ‘SeaIsle 1’, ‘SeaStar’, and ‘Salam’ (Table 6). It is similar to other commercial cultivars with medium to fine leaf texture and good turf density. UGP 73 was initially selected for its drought tolerance and has demonstrated superior performance in numerous non-irrigated trials as well as over a wide range of irrigation regimes (Table 8). It has excellent salt tolerance, similar to SeaStar (Table 11), and better performance under shade than SeaStar and ‘Platinum’ (Table 10). UGP 73 has good resistance to dollar spot disease, Clarireedia sp. (Tables 7, 12, and 13) and has been shown to produce acceptable quality sod with good tensile strength (Table 9).
7. Other: Golf course developments placed on coastal venues and problems associated with salinity are increasingly more prevalent issues in managed turfgrass. The trend for use of more salt-laden irrigation waters on turfgrass sites is expected to continue to rise and to further increase interest in developing more salt-tolerant grasses, especially halophytes such as seashore paspalum. A) ‘UGP 73’ has excellent turf quality over a wide range of mowing heights and has often outperformed Salam, SeaIsle 1, SeaStar, Platinum TE, and SeaDwarf (Tables 1, 8, 12, and 13). B) UGP 73 has excellent genetic color often better than Salam, SeaIsle 1, SeaStar, Platinum, and SeaDwarf. (Table 2, 5, 12, and 13) C) UGP 73 has demonstrated superior drought tolerance to SeaStar over a wide range of irrigation regimes (Table 8). D) The salt tolerance of UGP 73 is similar to that of SeaStar and better than that of SeaIsle 1 (Table 11). E) UGP 73 has demonstrated better tolerance to 60% shade than has Platinum and SeaStar (Table 10).

TABLE 1
NTEP 2016-2020 Seashore Paspalum-Turf Quality

| | | | | | | Top | 5 yr. |
|---|---|---|---|---|---|---|---|
| NTEP | 2016 | 2017 | 2018 | 2019 | 2020 | Statistical | Avg |
| Entry | (1-9)+ | (1-9) | (1-9) | (1-9) | (1-9) | Group | (1-9) |
| Salam | 6.1* | 6.1 | 6.0 | 6.0 | 6.2 | 2/5 | 6.1 |
| Sea Isle 1 | 6.2 | 6.0 | 6.1 | 6.2 | 5.9 | 4/5 | 6.1 |
| SeaStar | 5.9 | 6.1 | 6.1 | 6.1 | 6.0 | 4/5 | 6.0 |
| UGP 73 | 6.3 | 6.5 | 6.2 | 6.3 | 6.1 | 5/5 | 6.3 |
| C.V. | 4.4 | 1.3 | 2.6 | 2.1 | 3.0 | | |
| LSD | 0.4 | 0.1 | 0.2 | 0.2 | 0.3 | | |

+Rated on 1-9 scale with 6 = acceptable and 9 = excellent
*Means in the top statistical group are bolded.

TABLE 2
NTEP 2016-2020 Seashore Paspalum-Genetic Color

| | | | | | | Top | 5 yr. |
|---|---|---|---|---|---|---|---|
| NTEP | 2016 | 2017 | 2018 | 2019 | 2020 | Statistical | Avg. |
| Entry | (1-9) | (1-9) | (1-9) | (1-9) | (1-9) | Group | (1-9) |
| Salam | 5.7* | 6.2 | 6.2 | 6.6 | 5.9 | 3/5 | 6.1 |
| Sea Isle 1 | 6.0 | 6.4 | 6.3 | 6.7 | 5.5 | 2/5 | 6.2 |
| SeaStar | 5.0 | 6.4 | 6.6 | 6.3 | 6.3 | 1/5 | 6.1 |
| UGP 73 | 6.0 | 6.0 | 6.9 | 6.8 | 6.5 | 5/5 | 6.7 |
| C.V. | 4.5 | 2.8 | 2.5 | 4.4 | 5.7 | | |
| LSD | 0.4 | 0.3 | 0.3 | 0.5 | 0.6 | | |

*Means in the top statistical group are bolded.

TABLE 3
NTEP 2016-2020 Seashore Paspalum-Fall Density

| | Riverside, | Jay, | Miss. St., | Stillwater, | Top | Table |
|---|---|---|---|---|---|---|
| NTEP | CA | FL | MS | OK | Statistical | Avg. |
| Entry | (1-9) | (1-9) | (1-9) | (1-9) | Group | (1-9) |
| Salam | 6.2* | 6.4 | 6.8 | 6.4 | 4/5 | 6.4 |
| Sea Isle 1 | 5.4 | 6.6 | 7.0 | 6.0 | 3/5 | 6.2 |
| SeaStar | 5.2 | 6.5 | 6.8 | 6.3 | 2/5 | 6.1 |
| UGP 73 | 5.4 | 6.8 | 7.5 | 6.2 | 4/5 | 6.4 |
| C.V. | 8.7 | 9.0 | 6.1 | 2.9 | | 3.9 |
| LSD | 0.8 | 1.0 | 0.7 | 0.3 | | 0.4 |

*Means in the top statistical group are bolded.

TABLE 4
NTEP 2016-2020 Seashore Paspalum-Winter Quality

| | | | | | | Top | 5 yr. |
|---|---|---|---|---|---|---|---|
| NTEP | 2016 | 2017 | 2018 | 2019 | 2020 | Statistical | Avg |
| Entry | (1-9) | (1-9) | (1-9) | (1-9) | (1-9) | Group | (1-9) |
| Salam | 6.0 | 6.1 | 5.7 | 5.9 | 6.1 | 5/5 | 5.9 |
| Sea Isle 1 | 5.3 | 5.7 | 6.0 | 6.1 | 5.3 | 5/5 | 5.7 |
| SeaStar | 5.7 | 5.8 | 5.9 | 6.0 | 4.8 | 4/5 | 5.6 |
| UGP 73 | 5.7 | 5.8 | 5.8 | 5.9 | 5.1 | 4/5 | 5.6 |
| C.V. | 11.7 | 7.9 | 4.9 | 3.4 | 10.1 | | |
| LSD | 1.0 | 0.7 | 0.5 | 0.3 | 0.9 | | |

*Means in the top statistical group are bolded.

TABLE 5
NTEP 2016-2020 Seashore Paspalum-Color October

| | Auburn, | Jay, | Griffin, | Stillwater, | Top | Table |
|---|---|---|---|---|---|---|
| NTEP | AL | FL | GA | OK | Statistical | Avg. |
| Entry | (1-9) | (1-9) | (1-9) | (1-9) | Group | (1-9) |
| Salam | 5.9 | 6.3 | 6.2 | 6.2 | 1/4 | 6.2 |
| Sea Isle 1 | 5.8 | 6.1 | 6.3 | 6.0 | 1/4 | 6.1 |
| SeaStar | 6.1 | 5.9 | 6.2 | 5.7 | 2/4 | 6.0 |
| UGP 73 | 6.5 | 7.0 | 6.2 | 6.5 | 3/4 | 6.5 |
| C.V. | 7.6 | 7.0 | 6.0 | 4.1 | | 3.7 |
| LSD | 0.7 | 0.7 | 0.6 | 0.4 | | 0.4 |

*Means in the top statistical group are bolded.

TABLE 6
NTEP 2016-2020 Seashore Paspalum-Winter Kill

| | Fayetteville, |
|---|---|
| NTEP | AR |
| Entry | (% winter kill) |
| Salam | 22.5 |
| Sea Isle 1 | 20.8 |
| SeaStar | 56.7 |
| UGP 73 | 9.2 |
| C.V. | 22.0 |
| LSD | 11.6 |

*Means in the top statistical group are bolded.

TABLE 7
NTEP 2016-2022 Seashore Paspalum--Dollar Spot Disease

| | Auburn, | Jay, | Miss. St., | Table |
|---|---|---|---|---|
| NTEP | AL | FL | MS | Avg. |
| Entry | (1-9) | (1-9) | (1-9) | (1-9) |
| Salam | 2.0 | 4.3 | 7.0 | 4.8 |
| Sea Isle 1 | 2.7 | 7.0 | 7.3 | 5.7 |
| SeaStar | 5.3* | 8.3 | 4.7 | 6.1 |
| UGP 73 | 6.0 | 5.0 | 6.3 | 5.8 |
| C.V. | 55.4 | 23.9 | 19.4 | 18.7 |
| LSD | 4.2 | 2.6 | 1.8 | 1.7 |

*Means in the top statistical group are bolded

TABLE 8
SCRI Seashore Paspalum Drought
Trials-2017-College Station, Texas

| | Turf Quality (1-9) | | | | | | |
|---|---|---|---|---|---|---|---|
| SCRI | Irrigation Frequency (times/month) | | | | | | |
| Entry | 0 | 1 | 2 | 4 | 8 | Average | Rank |
| SeaStar | 5.49 | 6.22 | 6.14 | 6.93 | 6.84 | 6.32 | 3 |
| SR31-15-15 | 5.03 | 6.04 | 5.99 | 6.64 | 6.60 | 6.06 | 4 |
| UGP 144 | 5.58 | 6.24 | 6.30 | 6.89 | 6.77 | 6.35 | 2 |
| UGP 73 | 5.63 | 6.31 | 6.44 | 7.01 | 6.93 | 6.46 | 1 |

TABLE 9
Sod Handling Quality and Tensile Strength of Seashore
PaspalumOklahoma State University-2017

| | Mean | STS² | STS² | Mean |
|---|---|---|---|---|
| | SHQ¹ | 14 MAP | 18 MAP | STS² |
| Entry | (1-5) | (kg/dm) | (kg/dm) | (kg/dm) |
| Salam | 4.2* | 115.9 | 70.9 | 93.4 |
| SeaIsle 1 | 4.3 | 106.6 | 68.3 | 87.4 |
| SeaStar | 4.2 | 104.3 | 53.1 | 87.0 |
| UGP 73 | 4.2 | 89.6 | 105.1 | 97.1 |
| Test Average | 3.5 | 95.6 | 66.4 | 81.0 |
| (10 entries) | | | | |

¹Sod handling quality rated on 1-5 scale with 3 = minimum acceptable and 5 = excellent.
²Sod tensile strength in kg dm-2 recorded at the peak force required to cause sod pad tearing using a hand winch and force transducer/recorder.
*Means in the top statistical group are bolded.

TABLE 10
SCRI III Shade Trial-60% Shade-Griffin, Georgia 2020-2022

| | Turf Quality (1-9) | | |
|---|---|---|---|
| | Aug. 18, | Aug. 17, | 2-Yr. |
| Entry | 2021 | 2022 | Avg. |
| Platinum | 4.10 | 4.27 | 4.18 |
| SeaDwarf | 4.83 | 6.33 | 5.58 |
| SeaStar | 3.60 | 4.93 | 4.27 |
| UGP 73 | 5.83* | 5.70 | 5.77 |
| Test Average | 4.18 | 5.18 | 4.68 |
| (30 entries) | | | |

*Means in the top statistical group are bolded

TABLE 11
SCRI Greenhouse Salt Trial Seashore Paspalum-2015-Griffin,
GA-Final Data at 45 dS

| | | Visual | Visual | | |
|---|---|---|---|---|---|
| | Visual | Quality | Quality | Green | Total |
| SCRI | Quality | (% of | (% | Cover | Biomass |
| Entry | (1-9) | control) | reduction) | (%) | (grams) |
| SeaIsle 1 | 3.4 | 55.0 | 45.0 | 21.9 | 2.18 |
| SeaStar | 4.1* | 58.3 | 41.7 | 20.9 | 2.44 |
| UGP 73 | 5.0 | 57.9 | 42.1 | 31.1 | 2.35 |
| Test Avg. | 4.2 | 57.1 | 42.9 | 24.7 | 2.32 |
| (9 entries) | | | | | |

*Means in the top statistical group are bolded

TABLE 12
Summary of 2020 Advanced Seashore Trial-Fairway
(0.625 inch h.o.c.)-Griffin, GA

| | Cover | Turf | | | Dollar |
|---|---|---|---|---|---|
| | Yr 1 | Quality | Color | NDVI | Spot |
| Entry | (%) | (1-9) | (1-9) | (0-1) | (%) |
| Platinum | 93.3 | 7.2 | 7.3 | 0.71 | 1.0 |
| SeaDwarf | 86.7 | 6.8 | 7.6 | 0.70 | 3.0 |
| SeaStar | 90.0 | 7.1 | 7.9 | 0.71 | 15.0 |
| UGP 73 | 91.7 | 7.6 | 8.7 | 0.77 | 1.0 |
| Test Average | 80.9 | 7.3 | 7.9 | 0.70 | 4.5 |
| (30 entries) | | | | | |

*Means in the top statistical group are bolded

TABLE 13
Summary of 2020 Advanced Seashore Trial-Greens Height (0.125 inch h.o.c.)-Griffin, GA

| | Establishment | Green | Turf | | Fall | Winter | Seed | Dollar | | |
|---|---|---|---|---|---|---|---|---|---|---|
| | Cover | up | Quality | Color | Color | Color | Heads | Spot | NDVI¹ | Stimp |
| Entry | (%) | (1-9) | (1-9) | (1-9) | (1-9) | (1-9) | (1-9) | (%) | (0-1) | (ft.) |
| SeaDwarf | 76.2* | 8.2 | 6.8 | 6.0 | 6.4 | 6.4 | 6.5 | 5.1 | 0.64 | 7.2 |
| SeaIsle 2000 | 45.6 | 8.4 | 7.9 | 6.7 | 7.4 | 6.8 | 8.3 | 4.4 | 0.63 | 6.9 |
| SeaStar | 71.2 | 8.6 | 7.0 | 6.6 | 7.6 | 7.6 | 7.1 | 3.9 | 0.63 | 7.0 |
| Platinum | 60.0 | 8.5 | 7.1 | 6.8 | 7.5 | 7.4 | 8.1 | 4.8 | 0.64 | 6.9 |
| UGP 73 | 56.2 | 8.5 | 7.1 | 7.2 | 5.8 | 5.8 | 8.1 | 3.9 | 0.65 | 7.1 |
| Test Mean | 69.3 | 7.7 | 6.6 | 6.42 | 6.0 | 5.4 | 7.4 | 5.5 | 0.62 | 7.0 |
| 28 entries | | | | | | | | | | |

¹Normalized difference vegetative index is a measure of canopy density and color.
*Means in the top statistical group are bolded.

TABLE 14
Comparison of botanical characteristics of seashore paspalum cultivars.

| | | | | |
|---|---|---|---|---|
| Platinum | | | | |
| TE' | SeaDwarf' | SeaStar' | SeaIsle 1' | UGP 73' |

Characteristics of Flowering Tillers

| | | | | | |
|---|---|---|---|---|---|
| Length of peduncle (mm) | | | | | |
| Mean | 3.3 c¹ | 6.8 b | 4.2 bc | 9.5 a | 9.6 a |
| Std. Dev. | 2.8 | 1.7 | 2.7 | 2.9 | 5.1 |
| Diameter of peduncle (mm) | | | | | |
| Mean | 0.5 bc | 0.5 c | 0.5 c | 0.6 ab | 0.6 a |
| Std. Dev. | 0.1 | 0.1 | 0.1 | 0.1 | 0.1 |
| Length of longest spike (mm) | | | | | |
| Mean | 25.2 abc | 16.4 d | 21.5 c | 26.5 ab | 23.7 bc |
| Std. Dev. | 4 | 5 | 4 | 4.6 | 4.5 |
| Number of spikes per inflourescent | | | | | |
| Mean | 2 | 2 | 2 | 2 | 2 |
| Std. Dev. | 0 | 0 | 0 | 0 | 0 |
| Maxiumum number of spikes per inflourescent | | | | | |
| Mean | 2 | 2 | 2 | 2 | 2 |
| Std. Dev. | 0 | 0 | 0 | 0 | 0 |
| Length of spike branch from flag leaf (mm) | | | | | |
| Mean | 8.6 ab | 6.2 bcd | 5.3 cd | 7.8 abc | 8.9 ab |
| Std. Dev. | 2.2 | 2.3 | 2.5 | 3.7 | 5.20 |
| Florets per spike | | | | | |
| Mean | 16.2 bc | 15 c | 19.3 ab | 19.9 a | 15.7 c |
| Std. Dev. | 3 | 4 | 2.5 | 3.4 | 3.2 |
| Length of blade on flag leaf (mm) | | | | | |
| Mean | 18.9 a | 9.1 bc | 9 bc | 10.2 bc | 6 c |
| Std. Dev. | 4.9 | 2 | 2.4 | 3.5 | 2 |
| Width of blade on flag leaf (mm) | | | | | |
| Mean | 0.8 ab | 0.7 ab | 0.8 ab | 0.6 b | 0.9 a |
| Std. Dev. | 0.2 | 0.3 | 0.2 | 0.3 | 0.2 |
| Length of sheath on flag leaf (mm) | | | | | |
| Mean | 45.2 a | 26.7 cd | 29.6 bc | 44.5 a | 33.3 b |
| Std. Dev. | 5.5 | 6.8 | 6.5 | 8 | 4.3 |
| Length/width ratio of flag leaf blade (mm) | | | | | |
| Mean | 25.2 a | 17.1 bc | 12.3 cd | 19.0 abc | 6.9 d |
| Std. Dev. | 5.1 | 11.3 | 3.7 | 9.4 | 2.3 |
| Length of blade on 4th leaf (mm) | | | | | |
| Mean | 48.2 bc | 38.1 d | 43 cd | 56 ab | 37.4 d |
| S.D | 4.1 | 6.4 | 7.8 | 11.9 | 11.5 |
| Width of blade on 4th leaf (mm) | | | | | |
| Mean | 3.1 ab | 2.2 c | 3.2 a | 3 ab | 3.1 ab |
| Std. Dev. | 0.5 | 0.7 | 0.5 | 0.3 | 0.6 |
| Length of sheath on 4th leaf (mm) | | | | | |
| Mean | 14.6 abc | 12.3 c | 16.1 ab | 16.9 a | 14.1 bc |
| Std. Dev. | 2.1 | 3.5 | 1.6 | 2.5 | 2.6 |
| Length of 4th internode (mm) | | | | | |
| Mean | 11.6 a | 5.3 c | 7.8 cb | 11.3 ab | 11.3 ab |
| Std. Dev. | 3.1 | 1.6 | 2.1 | 2.8 | 4.1 |

Characteristics of Stolons

| | | | | | |
|---|---|---|---|---|---|
| Length of 4th internode (mm) | | | | | |
| Mean | 17.8 a | 12.1 b | 11.5 b | 14 b | 13.8 b |
| Std. Dev. | 4.8 | 2.3 | 1.5 | 3.3 | 4.1 |
| Diameter of 4th internode (mm) | | | | | |
| Mean | 2.1 a | 1.4 cd | 1.5 cd | 1.6 bc | 1.6 b |
| Std. Dev. | 0.2 | 0.2 | 0.2 | 0.2 | 0.2 |
| Length of 4th leaf blade (mm) | | | | | |
| Mean | 6.6 b | 4.3 bc | 4.7 bc | 9.2 a | 6.7 ab |
| Std. Dev. | 1.7 | 1.3 | 0.6 | 3.6 | 3.7 |
| Width of 4th leaf blade (mm) | | | | | |
| Mean | 2.6 a | 1.3 cd | 1.6 bc | 2.1 ab | 2.6 a |
| Std. Dev. | 0.6 | 0.5 | 0.4 | 0.7 | 0.7 |
| Length/width ratio of 4th leaf blade (mm) | | | | | |
| Mean | 2.6 c | 3.7 bc | 3.2 c | 4.9 a | 2.5 c |
| Std. Dev. | 0.8 | 1.6 | 0.8 | 2.1 | 0.9 |
| Length of leaf sheath on 4th node (mm) | | | | | |
| Mean | 8.3 a | 4.8 c | 6.6 b | 8.9 a | 8.6 a |
| Std. Dev. | 1.2 | 1 | 0.7 | 1.9 | 1 |

Other Characteristics

| | | | | | |
|---|---|---|---|---|---|
| Culm height (cm) | | | | | |
| Mean | 18.5 b | 5.5 e | 11.4 c | 22.3 a | 16.4 b |
| Std. Dev. | 2.9 | 1.9 | 1.4 | 3.3 | 2 |
| Seed head height (cm) | | | | | |
| Mean | 17.7 a | 2.9 d | 6.9 c | 17.4 a | 4.8 d |
| Std. Dev. | 2.3 | 0.9 | 0.9 | 2.7 | 1.9 |
| Color of upper leaf surface | | | | | |
| | 138 B² | 137 D | 137 D | 137 D | 137 B |
| Color of lower leaf surface | | | | | |
| | 137 A | 137 C | 137 C | 137 C | 137 C |
| Stolon Color | | | | | |
| | 146 C | 146 B | 146 D | 146 D | 146 D |
| Anther Color | | | | | |
| | 83 B | 83 B | 83 A | 83 A | 83 A |
| Stigma Color | | | | | |
| | 86 A | 86 A | 86 B | 86 B | 86 A |

¹Means within a row followed by the same letter are not considered statistically different according to Fisher's protected LSD at α = 0.05.
²Based on color codes of The Royal Horticultural Society ColourChart.

What is claimed is:

1. A new and distinct cultivar of the Paspalum vaginatum Swartz cultivar ‘UGP 73’ as illustrated and described herein.
